(19) United States
(12) Patent Application Publication
(10) Pub. No.: US 2010/0303908 A1
(43) Pub. Date: Dec. 2, 2010

(54) PHARMACEUTICAL COMPOSITION COMPRISING DESLORATADINE

(76) Inventors: Balamurugan Jeganathan, Tamil Nadu (IN); Ganesh Gat, Goa (IN); Jawed Hussain, Goa (IN)

Correspondence Address: SMITH PATENT CONSULTING, LLC, 515 East Braddock Road, Suite B, ALEXANDRIA, VA, 22314, US

(21) Appl. No.: 12/599,556

(22) PCT Filed: May 9, 2008
(86) PCT No.: PCT/EP2008/003764

§ 371 Date: Jan. 5, 2010

(30) Foreign Application Priority Data
May 11, 2007 (IN) 1002/CHE/2007

(51) Int. Cl. A61K 31/435; A61K 9/28; C07D 221/16; A61P 29/00; A61P 11/02

(52) U.S. Cl. 424/474; 514/290; 546/93

(57) ABSTRACT

The present application relates to pharmaceutical compositions comprising desloratadine and a pharmaceutically acceptable polymer.

The present invention relates to a pharmaceutical composition comprising desloratadine or a pharmaceutically acceptable salt thereof and a process for producing said composition.

Descarbonylethoxyloratadine, also called desloratadine, has the chemical name 8-chloro-6,11-dihydro-11-(4-piperdinylidene)-5H-benzo[5,6]cyclohepta[1,2-b]pyridine (further referred to as “desloratadine”, or “DCL”) and has the following chemical structure:

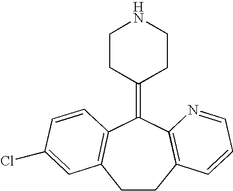

Desloratadine is a metabolic derivative of loratadine and is a long-acting tricyclic histamine antagonist with selective H₁-receptor histamine antagonist activity. Desloratadine is indicated for the relief of the nasal and non-nasal symptoms of allergic rhinitis (seasonal and perennial) in human patients 12 years of age and older. It is also indicated for the symptomatic relief of pruritus, reduction in the number of hives, and size of hives, in patients with chronic idiopathic urticaria, 12 years of age and older.

Several polymorphic forms of desloratadine are known, such as polymorph forms 1 and 2, as described in WO 99/01450 A1.

Desloratadine compositions were found to discolour in presence of acidic excipients such as lactose, lactose monohydrate and other mono- and di-saccharides, stearic acid, povidone and crospovidone when stored at a temperature of 40° C. at a relative humidity (“RH”) of 75%. This color instability of the active ingredient is attributed to a very minute amount of degradation product, in particular the N-formyl desloratadine, which is formed in the presence of a wide variety of the above mentioned excipients commonly used in oral formulations, especially tablet formulations.

U.S. Pat. No. 6,100,274 discloses a stable pharmaceutical composition containing desloratadine and a DCL-protective amount of a pharmaceutically acceptable basic salt such as calcium dibasic phosphate and an amount of at least one disintegrant to provide dissolution of at least about 80% by weight of the pharmaceutical composition in about 45 minutes, and which contains less than about 1% by weight of N-formyldescarbonyl-ethoxyloratadine. This document teaches to incorporated basic salt additives to reduce the degradation of active ingredients. However, the addition of such basic salt additives is less desirable because these may not be suitable for further pharmaceutical excipients and adjuvants to be added to the compositions, in particular acidic excipients, and therefore these basic salt additives limit the use of common excipients such as lactose in the compositions.

The commercially available CLARINEX 5 mg desloratadine tablets (Schering corporation) contain the excipients dibasic calcium phosphate dihydrate USP, micrystalline cellulose NF, corn starch NF, talc USP, carnauba wax NF, white wax NF, and a coating material consisting of lactose monohydrate, hydroxyl propyl methylcellulose, titanium dioxide, polyethylene glycol and FD&C Blue #2 aluminium lake, obviously following the teaching of the above US-patent.

EP 1614421 discloses stable pharmaceutical compositions of desloratadine which are formulated to avoid the incompatibility between the active ingredient and reactive excipients such as lactose and other mono- and bi-saccharides. The disclosed compositions include lactose-free, non-hygroscopic and anhydrous stable pharmaceutical compositions of desloratadine. The document teaches that stable compositions of desloratadine can be obtained by using anhydrous processes and excipients, such that the unbound water that may be present in the formulation is insufficient to initiate and/or accelerate any reaction of desloratadine with reactive excipients. Further means of stabilisation taught by this document include coating of desloratadine particles to avoid contact with reactive excipients, or using large particles of desloratadine, so that the surface area of the contact with the reactive excipients is reduced. However, these methods suggested in the document either provide solutions that involve very complex and therefore disadvantageous formulation procedures and avoid conventional formulation procedures, such as wet granulation, or the document suggests means that may effect bioavailability of the formulation, for example the coating of desloratadine particles or using large desloratadine particles.

WO2005/065047 A2 discloses a stable oral composition comprising a therapeutically effective amount of desloratadine and a stabilizer selected from the group comprising an antioxidant, a pharmaceutically acceptable organic compound that provides an alkaline pH, an alkali metal salt, or mixtures thereof, and pharmaceutically acceptable excipients. However, addition of said stabilizers, in particular the alkaline stabilizers, also limits the use of common excipients, in particular acidic excipients, in pharmaceutical compositions.

WO 2005/084674 discloses a pharmaceutical tablet of amorphous desloratadine which may also include PEG 3000. However, improved stability of the active ingredient in the obtained composition is not disclosed. Further methods how to obtain said composition is also not disclosed.

Therefore, there is still a need for a stable pharmaceutical composition comprising desloratadine that does not show the above prior art problems, and in particular wherein the choice of excipients contained is not limited and wherein in particular also acidic excipients can be used without deteriorating the stability of the active ingredient, i.e. the desloratadine.

It now has surprisingly been found that the above problems can be solved by addition of a pharmaceutical acceptable polymer, preferably a copolymer, in particular a block copolymer to pharmaceutical compositions comprising desloratadine to obtain a molecular mixture. Thereby in particular a solid pharmaceutical composition can be obtained, wherein common excipients and adjuvants, including acidic excipients, can be used and which solid pharmaceutical composition provides excellent stability of the contained active ingredient, i.e. desloratadine, in particular such stability that the content of N-formyl desloratadine in the solid pharmaceutical composition is less than about 1 wt-%, in particular less than about 0.5 wt-%, after storage for 3 months at 40° C. and 75% relative humidity.

Therefore the present application relates to a solid pharmaceutical composition comprising desloratadine or a pharmaceutical acceptable salt thereof as an active ingredient, wherein the composition comprises a pharmaceutically acceptable polymer, preferably a copolymer, in particular a block copolymer, which is most preferably a poloxamer and wherein the desloratadine or the pharmaceutically acceptable salt thereof and the pharmaceutically acceptable polymer are present in a molecular mixture.

The term “molecular mixture” according to the present invention means a mixture or blend on a molecular level. Thus the molecular mixture does not contain a significant amount of particles or zones containing substantially only one compound of the mixture and which are substantially free of the other compounds. Such a mixture can for example be identified therein that at least one parameter of the compounds is significantly different in the molecular mixture compared to the parameter of the compound in pure form (e.g. the pure amorphous or crystalline pure compound), such as X-ray diffraction, Raman spectroscopy, solution calorimetry, differential scanning calometry, solid state magnetic resonance spectra (ssNMR) or infra red spectroscopy. Each of these techniques is well known in the art. Such mixture can for example be identified by showing that the X-ray diffraction pattern of one compound, which was e.g. used in crystalline form and therefore showed a defined pattern as pure compound, can not be identified any more within the molecular mixture, but the mixture only shows a broad, undefined X-ray diffraction pattern. Preferably a molecular mixture is obtained by contacting and/or reacting compounds to be mixed in solution, as a molecular mixture can typically not be obtained in solid state mixtures. In solid state most molecules are fixed in the particles of the solid and are therefore not available to undergo molecular reactions. Preferred the molecular mixture is a complex of the compounds, i.e. the two components interact on a molecular level by weak interactions, such as ionic or Van der Waals interactions, and form more preferably a more or less stoichiometric molecular mixture.

The solid pharmaceutical compositions of the present application comprise desloratadine or a pharmaceutically acceptable salt thereof as active ingredient, preferably in an amount of about 0.1-10 wt-%, more preferably about 1-6 wt-%, in particular about 1.5-4 wt-%, based on the total weight of the composition. How to obtain desloratadine or the pharmaceutically acceptable salt thereof is known in the art. It has been found that if a crystalline form of desloratadine is used in mixture with a polymer, preferably a copolymer, in particular a block copolymer, as in the pharmaceutical compositions of the present application, crystallinity of the desloratadine can not be identified anymore, which is reflected therein that no x-ray powder diffraction peaks, corresponding to the crystalline desloratadine, can be detected anymore. Therefore the desloratadine is present in the pharmaceutical preparations of the present application in a non-crystalline form, i.e. the compositions of the present application are free from crystalline desloratadine or the pharmaceutically acceptable salt thereof.

The solid pharmaceutical composition of the present application may contain further active ingredients like nasal decongestants, e.g. pseudoephedrine sulphate. Pseudoephedrine (commonly abbreviated as PSE) is a sympathomimetic amine commonly used as a decongestant. The salts pseudoephedrine hydrochloride and pseudoephedrine sulfate are found either as single-ingredient preparations or more commonly in combination with antihistamines such as desloratadine. The further active ingredient is preferably present in an amount of 0.1-50 wt-%, based on the total weight of the composition. In such pharmaceutical compositions comprising desloratadine or a pharmaceutically acceptable salt thereof and a further active ingredient, such as pseudoephedrine sulphate, the desloratadine is preferably formulated as immediate release compound and the further active ingredient, such as pseudoephedrine sulphate, is preferably formulated as extended release using suitable rate controlling polymers in a controlled release matrix tablet. How to prepare such formulations is known to the person skilled in the art and is e.g. described in EP 1 110 543 A2.

In one embodiment the present application provides a method for stabilization of desloratadine by formation of complexes with the polymers, preferably the copolymers, in particular the block copolymers. Further, the present invention relates to a pharmaceutical composition comprising a desloratadine polymer complex, copolymer complex, or block copolymer complex, respectively, and other therapeutically active ingredients, such as pseudoephedrine, preferably formulated as bilayer tablets.

The therapeutically further “active ingredient” can further be one or more compounds or their acceptable salts, solvates, enantiomers or mixtures such as but not limited to: sympathomimetic nasal decongestants, analgesics, anti-inflammatory drugs such as non-steroidal anti-inflammatory drugs (NSAID), cox-2 inhibitors, anti-tussitives, bronchodilators and opoid analgesics. Such agents are well known in the art.

The solid pharmaceutical composition of the present application comprises at least one pharmaceutically acceptable polymer, preferably a copolymer, in particular a block copolymer. A polymer is a substance composed of repeating structural units or monomers, connected by covalent chemical bonds. A copolymer is a polymer obtained by polymerizing two (or more) different monomers. The copolymer can be alternating, periodic, random, statistical, or may be a block copolymer, which is preferred. A block copolymer is generally a copolymer consisting of blocks of repeating monomers, whereby the monomers differ from block to block.

Suitable pharmaceutically acceptable polymers, preferably copolymers, in particular block copolymers are known to the person skilled in the art. Preferably, the pharmaceutically acceptable polymer, preferably the copolymer, in particular the block copolymer is non-ionic. The block copolymer is preferably a “diblock copolymer”, a “triblock copolymer” or a “multiblock copolymer”. Most preferably, the block copolymer within the pharmaceutical compositions of the present application is a poloxamer, such as known by tradename PLURONICS, which are non-ionic block copolymers composed of a central hydrophobic chain of polyoxypropylene flanked by two hydrophilic chains of polyoxyethylene. By changing the length of the polymer blocks, the properties of such a poloxamer can be customized. Therefore, these polymers are commonly named with the word “poloxamer”, followed by a number to indicate which polymer is being discussed. Preferable poloxamers include poloxamer 124, poloxamer 188, poloxamer 237, poloxamer 338 and poloxamer 407. The block copolymers, in particular the poloxamers, are preferably contained within the pharmaceutical compositions of the present application in an amount of, but not limited to, about 0.1-20 wt-%, more preferably about 1-10 wt-%, even more preferably about 4-7 wt-%, such as about 5-6 wt-%, based on the total weight of the composition.

In one embodiment of the present application, the polymers in the pharmaceutical compositions of the present application are preferably polyethylene glycol (PEG) and polyethylene oxide (PEO). These are polymers composed of repeating oxyethylen subunits, i.e. non-ionic polymers with the structure HOCH₂(CH₂OCH₂)ₘCH₂OH. The two names, PEG and PEO, are chemically synonymous, but historically PEG has tended to refer to shorter polymers (200-35.000), PEO to longer polymers (335.000) of oxyethylene subunits. Such polymers are known by the names of Macrogols and tradenames such as Lutrol, or Polyox P56 and P50. By changing the length of the polymer, the properties of such polymer can be customized. Therefore, these polymers are commonly named with the word “PEG”, followed by a number to indicate which polymer is being discussed. Preferable PEGs include but not limited to PEG 1000, PEG 2000, PEG 3000, PEG 4000 and PEG 6000. While PEG and PEO with different molecular weights find use in different applications and have different physical properties (e.g. viscosity) due to chain length effects, their chemical properties are nearly identical. The polymers, in particular the PEGs and PEOs, are preferably contained within the pharmaceutical compositions of the present application in an amount of, but not limited to, about 0.1-20 wt-%, more preferably, about 1-10 wt-%, in particular about 4-7 wt-%, such as about 6 wt-%, based on the total weight of the composition.

In one embodiment of the present invention the solid pharmaceutical composition does not contain a PEG, in particular no PEG 3000.

The solid pharmaceutical composition of the present application may contain monosaccharides and/or disaccharides, and optionally also an acidifying agent, if necessary or desired. Due to the use of a polymer, preferably a copolymer, in particular a block copolymer in combination with the desloratadine within the pharmaceutical compositions of the present application said saccharides and/or acidifying agents can be added to the pharmaceutical compositions of the present application without deteriorating the stability of the desloratadine in the composition.

The solid pharmaceutical composition of the present application may further contain one or more excipients and/or adjuvants which are commonly used in such formulations, preferably selected from the group of fillers, disintegrants, binders, antiadherents and lubricants. Preferably, the pharmaceutical composition of the present application comprises about 0.1-10 wt-% of the active ingredient, more preferably about 0.5-5 wt-%, in particular about 1-4 wt-%, such as about 2 wt-% of the active ingredient, about 0.1-20 wt-%, more preferably about 2-10 wt-%, in particular about 5-7 wt-% of a polymer, preferably a copolymer, in particular a block copolymer, about 5-80 wt-%, preferably about 10-60 wt-% of a filler, about 5-70 wt-%, preferably about 20-70 wt-%, such as about 70 wt-% of a disintegrant, about 0-20 wt-%, more preferably about 5-18 wt-%, in particular about 8 wt-% of a binder, about 0-10 wt-%, preferably about 2-8 wt-%, in particular about 5 wt-% of an antiadherent and about 0-3 wt-%, preferably about 1-2 wt-% of a lubricant, each based on the total weight of the composition. The further common excipients and adjuvants are present in an amount such that the total weight of the pharmaceutical compositions of the present application results in 100 wt-%.

As filler, on or more components can be used which constitute a part of the pharmaceutical composition of the present application to reach the necessary total mass of the pharmaceutical composition. Preferable fillers are sugars or sugar analogues and derivatives thereof, in particular lactose, such as lactose monohydrate or water-free lactose, dextrose, sorbit, mannit, saccharose, maltodextrin, isomalt and tablettose, whereby lactose monohydrate, in particular in granular form, is especially preferred. Celluloses like microcrystalline cellulose can also be used as fillers within the pharmaceutical compositions of the present application. Preferably, the pharmaceutical compositions according to the present application comprise about 5-80 wt-%, more preferably about 10-60 wt-%, in particular about 10-40 wt-% of a filler, based on the total weight of the composition.

As disintegrants to be used in the pharmaceutical composition of the present application, agar agar, alginic acid, calcium carbonate, microcrystalline cellulose, croscarmellose sodium, crospovidone, sodium starch glucolate, starches and modified starches such as pregelatinized starch, clays, other algines or other celluloses, gums or mixtures thereof can be exemplified. Microcrystalline cellulose and croscarmellose sodium are the preferred disintegrants to be used within the pharmaceutical compositions of the present application. Preferably, the pharmaceutical compositions according to the present application comprise about 5-70 wt-%, preferably about 10-70 wt-%, in particular about 20-70 wt-% of a disintegrant, based on the total weight of the composition.

As binders, i.e. a compound enabling granulation of the active ingredient and further excipients and adjuvants into granules, to be used in the pharmaceutical composition of the present application, starches, in particular corn starch, potato starch, or modified starches, such as pregelatinized starch, which is preferred, gelatine, natural and synthetic gums such as acacia, sodium alginate, alginic acid, or other alginates, guargum, celluloses and its derivatives, such as ethylcellulose, cellulose acetate, carboxymethylcellulose calcium, sodiumcarboxymethylcellulose, methylcellulose, hydroxypropylmethylcellulose, microcrystalline cellulose, polyvinylpyrrolidone, copovidone, or mixtures thereof can be exemplified. Preferably, pregelatinized starch is used as the binder of the pharmaceutical composition of the present application. The binder is usually present in an amount of about 0-20 wt-%, preferably about 2-19 wt-%, in particular about 5-18 wt-%, based on the total weight of the composition.

As antiadherent to be used in the pharmaceutical composition of the present application, calciumsilicate, magnesiumsilicate, siliciumdioxide, colloidal siliciumdioxide, talc, or mixtures thereof can be exemplified. Preferably, a pharmaceutical composition according to the present application contains about 0-10 wt-%, more preferably about 2-8 wt-%, in particular about 5 wt-% of an antiadherent, based on the total weight of the composition.

As lubricant to be used in the pharmaceutical compositions of the present application fatty acids or fatty acid derivatives, such as alkali and earth alkali salts of stearic, lauric and/or palmitic acid are exemplified, in particular calcium stearate, magnesium stearate, sodium stearyl fumarate, sodium lauryl sulfate, talc, hydrogenated vegetable oil, zinc stearate, or mixtures thereof. Preferably, the lubricant is present in the pharmaceutical compositions according to the present application in an amount of about 0-3 wt-%, based on the total weight of the composition.

The solid pharmaceutical composition of the present application may further contain an acidifying agent, such as citric acid, tartaric acid, fumaric acid, malic acid, or salts and hydrates thereof, e.g. citric acid monohydrate, or any other suitable acidifying agent. The acidifying agent is present in the pharmaceutical compositions according to the present application in an amount of about 0-5 wt.-%, preferably about 0.1-4 wt.-%, in particular about 1-3 wt.-%, based on the total weight of the composition.

In one embodiment of the present application the solid pharmaceutical compositions do not comprise a basic stabilizer of the active ingredient, i.e. desloratadine, in particular the pharmaceutical compositions do not comprise dibasic calcium phosphate.

Preferably, the solid pharmaceutical composition according to the present application contains less than about 1 wt-%, in particular less than about 0.5 wt-% of N-formyl desloratadine, based on the total weight of the composition. In particular, the solid pharmaceutical composition according to the present invention is such that when subjected to stressed conditions, i.e. at 40° C. at 75% relative humidity for a longer period of time, such as 3 months, the amount of N-formyl desloratadine contained is still less than about 1 wt-%, preferably less than about 0.5 wt-%, even more preferably less than about 0.25 wt-%, such as about 0.1 wt-% or less, based on the total weight of the composition.

The present application also relates to a solid pharmaceutical composition which comprises desloratadine or a pharmaceutically acceptable salt thereof as active ingredient and which does not contain desloratadine or the pharmaceutically acceptable salt thereof in crystalline form or in amorphous form. In particular, no X-ray diffraction pattern and/or any other parameter, such as IR-spectrum, NMR spectrum or further measurable parameter of the corresponding crystalline form or amorphous form of the desloratadine can be detected in such composition.

The pharmaceutical composition of the present application is in solid form, and is more preferably a tablet, in particular a film coated tablet or an orally disintegrating tablet (fast dissolving/dispersible tablet), which can be obtained by methods known in the art, such as tablettation by granulation. Wet granulation is the most preferred method to obtain the tablet of the present application.

Preferably, the tablet, in particular the film coated tablet, according to the present application has a dissolution profile such that 80 wt-% or more of the active ingredient, in particular the desloratadine, contained in the tablet, is dissolved within 45 minutes.

The pharmaceutical composition according to the present invention comprising desloratadine or a pharmaceutically acceptable salt thereof is a solid pharmaceutical composition, which is any solid dosage form known to the person skilled in the art, such as dragees, satchets, capsules, and is preferably in form of a tablet which is more preferably coated, in particular film coated, with one or more coating materials. The coating materials are not particularly limited and are known to the person skilled in the art. Examples of coating agents are hydroxypropylmethylcellulose (HPMC) based film-coatings. However, the coating materials should be selected such that the dissolution profile and the bioavailability of the active ingredient are not deteriorated.

In the present application, the term “dissolution profile” refers to the time trace of the amount of active ingredient, in particular desloratadine, which is dissolved, based on the total amount of active ingredient, which is contained within the solid pharmaceutical composition, which is preferably a tablet. Such a dissolution profile can be obtained by dissolving the solid pharmaceutical composition, which is preferably a tablet in an USP-apparatus II in 900 ml 0.1 M hydrochloric acid at 37° C. at a stirring speed of 50 rpm and the amount of active ingredient dissolved is measured over a defined period of time, e.g. 60 minutes at distinct time points, e.g. every 15 minutes.

Preferably, the solid pharmaceutical composition of the present application is preferably obtained by the process according to the present invention, to obtain a solid pharmaceutical composition. Preferably, the process for the preparation of the solid pharmaceutical composition of the present application, which is a tablet, comprises the steps of

a) adding the active ingredient, i.e. desloratadine or a pharmaceutically acceptable salt thereof and optionally a further active ingredient, to a solution of the pharmaceutically acceptable polymer, preferably the copolymer, in particular the block copolymer, in particular an aqueous solution, preferably to obtain a solution of desloratadine or a pharmaceutically acceptable salt thereof and of the pharmaceutically acceptable polymer,

b) optionally adding pharmaceutically acceptable excipients and adjuvants,

c) drying said mixture,

d) optionally adding further pharmaceutically acceptable excipients and adjuvants,

e) preparing a solid pharmaceutical dosage form, in particular a tablet.

The obtained tablet can be further coated, in particular film coated.

In one embodiment of the present application, suitable solvents other than aqueous solvents can also be used to dissolve the polymer, preferably the copolymer, in particular the block copolymer and the desloratadine in step a) of the above described process, i.e. in the preparation of the desloratadine complex.

Preferably, the process for the preparation of the solid pharmaceutical composition according to the present invention is carried out such that the polymer, preferably the copolymer, in particular the block copolymer, which is preferably a poloxamer, in particular the poloxamers listed above, is dissolved in suitable solvents, preferably aqueous solvents, in particular water, to get a clear solution, adding the active ingredient, i.e. desloratadine and optionally a further active ingredient, to said solution and mixing. Optionally, pharmaceutically acceptable excipients and adjuvants can already be added to said solution/dispersion, such as citric acid monohydrate, followed by further stirring to get a molecular mixture, i.e. a complex of desloratadine and the polymer. The addition of the pharmaceutically acceptable excipients and adjuvants and the drying step could be such that optionally sieved and mixed pharmaceutically acceptable excipients and adjuvants, such as celluloses and/or starches or their derivatives, are mixed in a suitable mixer and the solution of desloratadine and the polymer, preferably copolymer, in particular block copolymer is adsorbed over the powder mixture of the pharmaceutically acceptable excipients and adjuvants followed by granulation. The wet mass, i.e. the wet granules, can be dried in a fluid bed drier and sieved afterwards. Optionally, further pharmaceutically acceptable excipients and adjuvants can then be added to said dried granules, including e.g. addition of talc to the above blend as optional lubrication step, preferably followed by compression of the lubricated blend into a solid pharmaceutical dosage form, in particular a tablet, which can optionally be film coated.

In one embodiment of the present application the solid pharmaceutical compositions do not contain powder or particles of substantially pure desloratadine or a pharmaceutically acceptable salt thereof, which do in particular not comprise the pharmaceutically acceptable polymer, or which have not been solved and mixed with a solution of the pharmaceutically acceptable polymer to obtain a molecular mixture of the desloratadine or the pharmaceutically acceptable salt thereof and the pharmaceutically acceptable polymer.

The present invention further relates to the solid pharmaceutical compositions which can be obtained by the above described process to obtain a solid pharmaceutical composition.

The present invention also relates to the use of a pharmaceutically acceptable polymer, preferably copolymer, in particular block copolymer, such as a poloxamer, as defined above, in a pharmaceutical composition according to the present application.

The present application provides solid pharmaceutical compositions, which show improved stability of the desloratadine, in particular in the presence of lactose, whereby the improved stability is shown by reduced formation of impurities, in particular N-formyl desloratadine, during storage, in particular storage at 40° C. and 75% relative humidity for a storage time of 1-3 months. The presence of the stabilizer, i.e. the polymer, preferably copolymer, in particular block copolymer, such as poloxamer, seems to inhibit the formation of the impurities, even in the presence of reactive acidic excipients, such as lactose. This could be explained by formation of a complex of desloratadine and the polymer, preferably copolymer, in particular block copolymer, which complex stabilises the desloratadine in the compositions, leading to improved stability of the active ingredient compared with compositions known in the art.

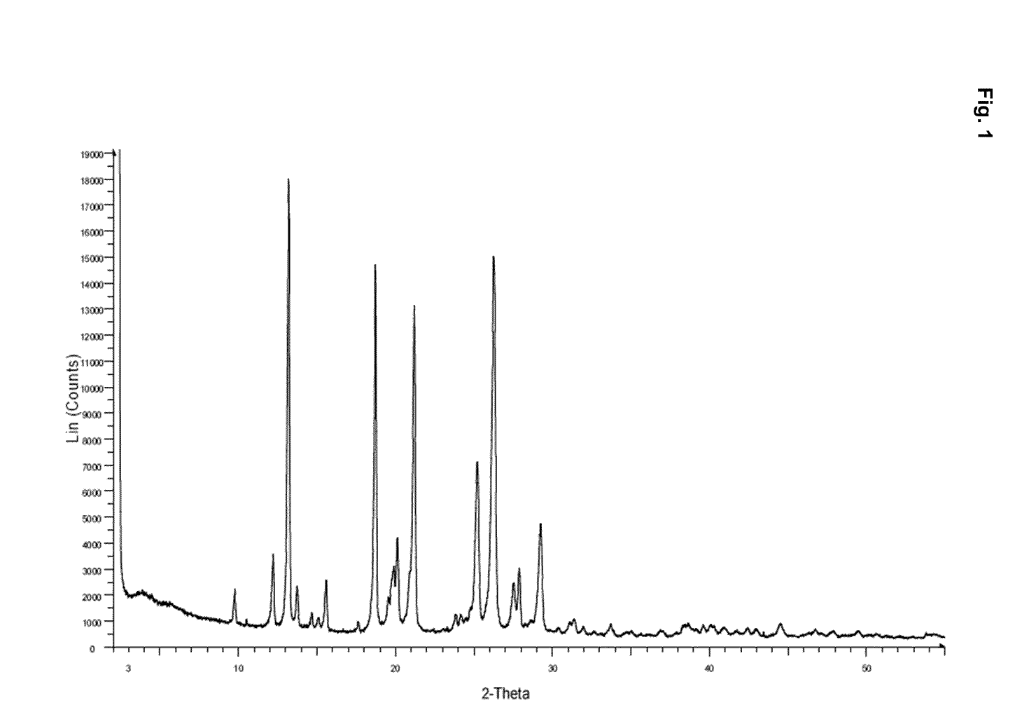

FIG. 1 shows an X-ray powder diffraction pattern of crystalline desloratadine.

(Type: 2Th/Th locked—Start 2.000°—End: 55.000°—Step: 0.016°—Step time: 74.8 s—Temp: 25° C. (room) —Time started: 12 s —2—Theta: 2.000°—Theta: 1.000°—Chi—0.00°—Phi: 0.00°—X: 0.0 mm—Y: 0.0 mm)

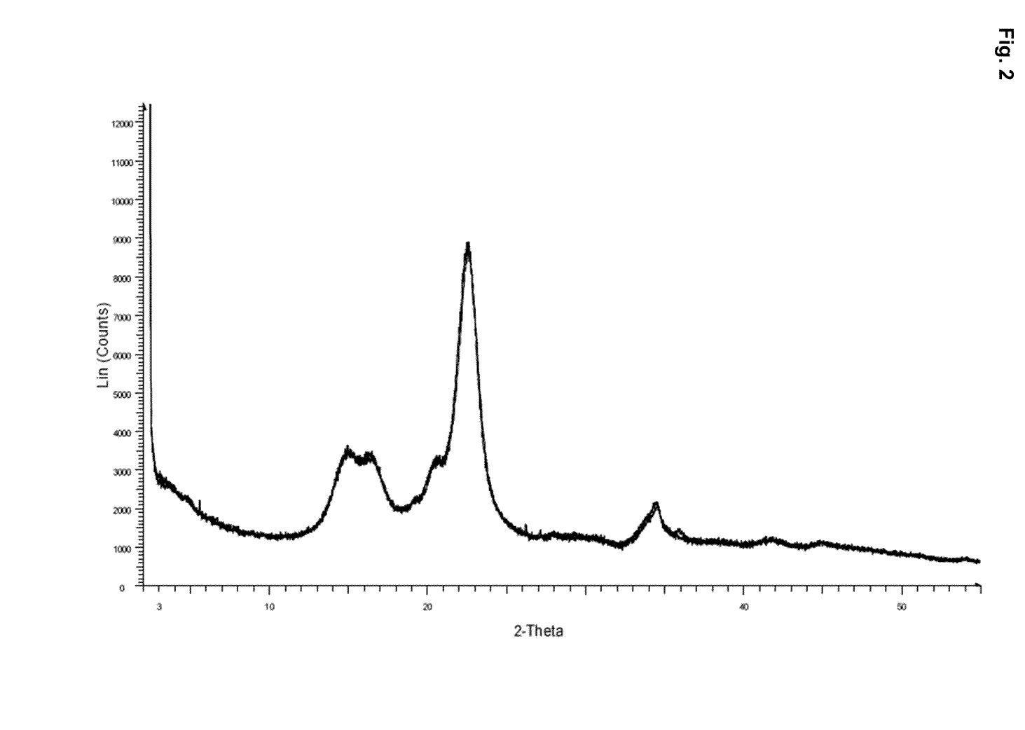

FIG. 2 shows an overlay of two X-ray powder diffraction patterns of poloxamer and desloratadine-poloxamer complex. (Parameters as in FIG. 1).

The following examples are not intended to be limiting:

### EXAMPLE 1

Desloratadine tablet with a block copolymer (poloxamer)

| Ingredients | Mg/tablet |
|---|---|
| Desloratadine | 5.0 |
| Poloxamer | 15.0 |
| Citric acid monohydrate | 6.5 |
| Cellulose, microcrystalline | 85.0 |
| Starch, pregelatinized | 20.0 |
| Cellulose, microcrystalline | 76.5 |
| Croscarmellose sodium | 13.0 |
| Lactose monohydrate, granular | 26.0 |
| Talc | 13.0 |
| Total | 260.0 |

The tablet was produced according to the following procedure:

1. Weighed quantity of block copolymers was dissolved in a sufficient amount of water to get a clear solution.

2. Desloratadine was dispersed in the polymer solution of step 1 and mixed.

3. To the dispersion/solution of step 2, citric acid monohydrate was added under stirring conditions and stirred well for about 30 minutes to get a complex of desloratadine and the polymer.

4. Cellulose and starch were sieved through # 30 mesh and mixed well in a polybag.

5. The desloratadine complex of step 3 was absorbed over the powder mixture of step 4, followed by granulation.

6. The wet granules obtained in step 5 was dried in a fluid bed drier.

7. The dried granules obtained in step 6 were sized through # 30 mesh.

8. The further excipients, i.e. the extra granular material was weighed, sieved and mixed for about 5 minutes with the dried granules obtained in step 7.

9. Talc was sieved through # 60 mesh and added to the above blend, followed by lubrication for 2 minutes.

10. Lubricated blend obtained in step 9 was compressed into tablets, which were film coated afterwards using HPMC based coating materials.

### EXAMPLE 2

Desloratadine tablets with a polymer (PEG)

| Ingredients | Mg/tablet |
|---|---|
| Desloratadine | 5.0 |
| Citric acid monohydrate | 2.5 |
| Polyethylene glycol (PEG 6000) | 10.0 |
| Cellulose, microcrystalline | 90.0 |
| Starch, pregelatinized | 31.0 |
| Croscarmellose sodium | 2.5 |
| Talc | 5.0 |
| Opadry II Blue (coating material) | 4.0 |
| Total | 150.0 |

The tablet was prepared as described in Example 1.

### EXAMPLE 3

Desloratadine tablets with a block copolymer (poloxamer)

| Ingredients | Mg/tablet |
|---|---|
| Desloratadine | 5.0 |
| Poloxamer | 7.5 |
| Citric acid monohydrate | 2.5 |
| Copovidone | 5.0 |
| Cellulose, microcrystalline | 67.0 |
| Starch, pregelatinized | 20.0 |
| Croscarmellose sodium | 11 |
| Lactose monohydrate, granular | 15.0 |
| Talc | 4.0 |
| Opadry II Blue (coating material) | 3.0 |
| Total | 140.0 |

The tablet was prepared as described in Example 1.

### EXAMPLE 4

Desloratadine tablets with a block copolymer (poloxamer)

| Ingredients | Mg/tablet |
|---|---|
| Desloratadine | 5.0 |
| Poloxamer | 7.5 |
| Citric acid monohydrate | 2.5 |
| Cellulose, microcrystalline | 51.0 |
| Starch, pregelatinized | 20.0 |
| Cellulose, mycrocrystalline | 16.0 |
| Croscarmellose sodium | 11.0 |
| Lactose monohydrate, granular | 15.0 |
| Talc | 4.0 |
| Opadry II Blue (coating material) | 3.0 |
| Total | 135.0 |

The tablet was prepared as described in Example 1.

### EXAMPLE 5

Desloratadine tablets with a block copolymer (poloxamer)

| Ingredients | Mg/tablet |
|---|---|
| Desloratadine | 5.0 |
| Poloxamer | 7.5 |
| Citric acid monohydrate | 2.5 |
| Cellulose, microcrystalline | 51.0 |
| Starch, pregelatinized | 20.0 |
| Hypromellose (6 cps) | 4 |
| Cellulose, mycrocrystalline | 21.0 |
| Croscarmellose sodium | 2.0 |
| Lactose monohydrate, granular | 15.0 |
| Talc | 4.0 |
| Opadry II Blue (coating material) | 3.0 |
| Total | 135.0 |

The tablet was prepared as described in Example 1.

Example 6 (Comparative)

Desloratadine tablet without poloxamer

| Ingredients | Mg/tablet |
|---|---|
| Desloratadine | 5.0 |
| EDTA DISODIUM | 3.0 |
| Dicalciumphosphate dihydrate | 60.0 |
| Cellulose, microcrystalline | 45.0 |
| Starch 1500 | 22.5 |
| Starlac | 10.0 |
| Talc | 4.5 |
| Total | 150.0 |

The desloratadine tablet without poloxamer was manufactured by the following process:

1. The desloratadine, pregelatinized starch (Starch 1500) and the EDTA disodium were sieved through an ASTM mesh # 30 sieve and mixed in a polybag.

2. The dicalciumphosphate dihydrate, cellulose and starlac were sieved through an

ASTM mesh # 30 sieve and added to the powder blend obtained in step 1 and mixed in a polybag to get a uniform powder mixture

3. Talc was sieved through an ASTM mesh # 40 mesh and added into the powder blend obtained in step 2 and mixed in a polybag to get a uniform lubricated powder blend.

4. The blend obtained in step 3 was compressed into tablets using 6.5 mm round tooling.

5. The tablets were coated further using opadry II blue coating materials.

### EXAMPLE 7

The desloratadine tablets prepared as per the above example 1 and comparative example 6 were subjected to a stability study by storing in closed high density polyethylene (HDPE) bottles at 40° C. and 75% relative humidity (RH) for 3 months. The impurity profile of the tablets during the stability studies is shown in the following table 1:

TABLE 1
Comparison of stability study results @ 40° C./75% RH

| | | | After 1 | After 2 | After 3 |
|---|---|---|---|---|---|
| Attributes | Impurities | Initial | month | months | months |
| Example 1 | N-formyl | <0.01% | <0.01% | 0.02% | 0.03% |
| (with | Desloratadine | | | | |
| poloxamer) | | | | | |
| Comparative | N-formyl | 0.01% | 0.08% | 0.19% | 0.30% |
| Example 6 | Desloratadine | | | | |
| (Sample | | | | | |
| without | | | | | |
| polymer) | | | | | |

Additionally, the following stability study comparing the composition of Example 1 of the present application with the commercial reference product AERIUS® (desloratadine) 5 mg tablets (Essex Pharma) have been conducted using the same conditions. The impurity profiles obtained from said comparison is given in Table 2 below:

TABLE 2
Impurity profiles (@ 40° C./75% RH)

| | | | After 1 | After 2 | After 3 |
|---|---|---|---|---|---|
| Attributes | Impurities | Initial | month | months | months |
| Example 1 | N-formyl | <0.01% | <0.01% | 0.02% | 0.03% |
| (Composition | desloratadine | | | | |
| with poloxamer) | | | | | |
| AERIUS ® 5 mg | N-formyl | 0.06% | 0.12% | 0.23% | 0.33% |
| tablets | desloratadine | | | | |
| (Reference product) | | | | | |
| Batch No. | | | | | |
| 5STBAA2B02 | | | | | |

These experiments show improved stability of the active ingredient desloratadine within the pharmaceutical compositions of the present application.

### EXAMPLE 8

Two samples of tablets according to the invention containing 5 mg desloratadine were stored at 25° C. and 60% RH and 40° C. and 75% RH, respectively. For stability testing the tablets were analyzed by means of X-ray powder diffraction after 2 and 3 months.

No peaks which can be attributed to crystalline desloratadine were detected in the XRPDs of the tablet samples. This confirms the good stability of the non-crystalline form of the active ingredient in the pharmaceutical composition of the present invention.

1. A solid pharmaceutical composition comprising desloratadine or a pharmaceutically acceptable salt thereof as active ingredient, characterized in that the composition further comprises a pharmaceutically acceptable polymer, wherein the desloratadine or the pharmaceutically acceptable salt thereof and the pharmaceutically acceptable polymer are present in a molecular mixture.

2. The solid pharmaceutical composition according to claim 1, wherein the pharmaceutically acceptable polymer is non-ionic.

3. The solid pharmaceutical composition according to claim 1, wherein the pharmaceutically acceptable polymer is a copolymer.

4. The solid pharmaceutical composition according to claim 1, wherein the pharmaceutically acceptable polymer is a block copolymer.

5. The solid pharmaceutical composition according to claim 4, wherein the pharmaceutically acceptable block copolymer is a poloxamer.

6. The solid pharmaceutical composition according to claim 1, wherein the pharmaceutically acceptable polymer is polyethylene glycol (PEG) or polyethylene oxide (PEO).

7. The solid pharmaceutical composition according to claim 1, wherein said composition which-further comprises a monosaccharide and/or a disaccharide.

8. The solid pharmaceutical composition according to claim 1, wherein said composition which-further comprises an acidifying agent.

9. The solid pharmaceutical composition according to claim 1, wherein said composition further comprises pseudoephedrine sulphate.

10. The solid pharmaceutical composition according to claim 1, wherein said composition further comprises one or more excipients and/or adjuvants selected from the group of fillers, disintegrants, binders, antiadherents and lubricants.

11. The solid pharmaceutical composition according to claim 10, wherein said composition comprises:
about 0.1-10 wt-% of the active ingredient,
about 0.1-20 wt-% of the block copolymer,
about 5-80 wt-% of a filler,
about 5-70 wt-% of a disintegrant,
about 0-20 wt-% of a binder,
about 0-10 wt-% of an antiadherent,
about 0-3 wt-% of a lubricant,
wherein each of the above weight percentages is based on the total weight of the composition.

12. The solid pharmaceutical composition according to claim 1, wherein said composition contains less than about 1 wt-% of N-formyl desloratadine, based on the total weight of the composition.

13. The solid pharmaceutical composition according to claim 1, wherein said composition contains less than about 0.5 wt-% of N-formyl desloratadine, based on the total weight of the composition.

14. The solid pharmaceutical composition according to claim 1, wherein said composition contains less than about 1 wt.-% of N-formyl desloratadine, based on the total weight of the composition, after a storage for 3 months at 40° C. and 75% relative humidity.

15. The solid pharmaceutical composition according to claim 1, wherein said composition is in the form a tablet.

16. The solid pharmaceutical composition according to claim 15, wherein said tablet is a film coated tablet.

17. The solid pharmaceutical composition according to claim 15, characterized in that it shows a dissolution profile such that 80 wt-% or more of the desloratadine or a pharmaceutically acceptable salt thereof contained in the composition is dissolved within 45 minutes.

18. A solid pharmaceutical composition comprising desloratadine or a pharmaceutically acceptable salt thereof as active ingredient, characterized in that the composition does not contain desloratadine or the pharmaceutically acceptable salt thereof which is in crystalline or amorphous form.

19. A process for the preparation of a pharmaceutical composition according to claim 15, said process comprising the steps of:
a. adding desloratadine or a pharmaceutically acceptable salt thereof to a solution of a pharmaceutically acceptable polymer,
b. optionally adding pharmaceutically acceptable excipients and adjuvants,
c. drying of said mixture,
d. optionally adding further pharmaceutically acceptable excipients and adjuvants,
e. preparing a solid pharmaceutical dosage form.

20. The process according to claim 20, wherein the dosage form is a tablet which is optionally further film coated.

21. A solid pharmaceutical composition obtained by a process according to claim 19.

22. A method for the treatment of nasal or non-nasal symptoms of allergic rhinitis in a subject in need thereof, said method comprising the step of administering to said subject an effective amount of the solid pharmaceutical composition according to claim 1, wherein said pharmaceutically acceptable polymer is a copolymer or block copolymer.

23. A solid pharmaceutical composition comprising desloratadine or a pharmaceutically acceptable salt thereof as active ingredient, characterized in that the composition comprises a pharmaceutically acceptable polymer, copolymer, or block copolymer, further wherein said composition contains less than about 0.5 wt-% of N-formyl desloratadine, based on the total weight of the composition.

24. A solid pharmaceutical composition comprising desloratadine or a pharmaceutically acceptable salt thereof as active ingredient, characterized in that the composition comprises a pharmaceutically acceptable polymer, copolymer, or block copolymer, further wherein said composition contains less than about 1 wt-% of N-formyl desloratadine, based on the total weight of the composition, after a storage for 3 months at 40° C. and 75% relative humidity.
